(19) United States
(12) Patent Application Publication
(10) Pub. No.: US 2022/0362514 A1
(43) Pub. Date: Nov. 17, 2022

(54) URETHRAL CATHETER CAPABLE OF SMOOTHLY PASSING THROUGH URETHRAL STENOSIS SECTION

(71) Applicant: WUHAN XUNSHU TECHNOLOGY CO., LTD., Hubei (CN)

(72) Inventors: Guxi Xiang, Hubei (CN); Zhiwei Ouyang, Hubei (CN)

(21) Appl. No.: 17/620,177

(22) PCT Filed: Jun. 25, 2019
(86) PCT No.: PCT/CN2019/092726

§ 371 Date: Dec. 17, 2021

(51) Int. Cl. A61M 25/00

(52) U.S. Cl. CPC A61M 25/0074; A61M 25/0017; A61M 25/0097; A61M 2025/0681

(57) ABSTRACT

A urethral catheter. A cavity is formed at the head end of the urethral catheter, and an internal span of the cavity is greater than that of an opening. A check device (2) located at the front end of a guide core (5) is received in the cavity, and the check device (2) is a guide core (5) that can be retracted into a guide core catheter (4) and can move within the guide core catheter (4). The guide core (5) can expand outward after moving out of the guide core catheter (4). The span of the top of the expanded guide core (5) is greater than the outer diameter of the opening of the cavity at the head end of the urethral catheter. The urethral catheter eliminates a guide extension inserted inside the urethral catheter, and avoids any structural parts provided in the front end of the urethral catheter, and no accessory device is provided at the inner side of the catheter wall of the conventional urethral catheter, so that the internal urination cross-sectional area of the urethral catheter is the same as the area of the inner holes of the urethral catheter, thereby greatly increasing the urine output of the urethral catheter and greatly reducing the injection resistance of a lubricant.

### FIELD OF THE INVENTION

The present invention relates to a catheter, in particular to a catheter which can be easily inputted into a urethra of patients, especially can be operated to smoothly pass through the narrowest section of the urethra of patients, who suffered from stricture of prostate, or prostatic hyperplasia, etc.

### PRIOR ART

In prior art, in order to prevent a guiding core from penetrating out of a tube wall of a catheter, a complex structure is arranged. For example, a size of a hole for draining urine is reduced to be less than a diameter of the guiding core; a front end of the catheter is made to have a section of rigid cylinder wall; or a limiting means is set at a front section of the guiding core, etc., so as to prevent the guiding core from penetrating through the hole for discharging urine.

Those technical solutions in the prior art have the same problems as each other, i.e., the cross-sectional area for discharging urine within the catheter tube has to become smaller; a catheter's capacity for discharging urine, i.e. a maximum volume of the discharged urine per minute, has to be reduced; a resistance for injecting a lubricant is increased; and any patient to use the catheter is far from being comfortable.

### SUMMARY OF THE INVENTION

The object of the invention is to provide a catheter, which is strong for discharging urine, is able to discharge a large volume of urine, has a low resistance to inject lubricant into the catheter, and make any patient for using the catheter to be comfortable.

Thus, according to one aspect of the invention, there is provided a catheter, wherein a head end of the catheter is provided with a cavity, an internal span of the cavity is greater than its mouth span, and the cavity accommodates a check means located at a front end of a guiding core.

Preferably, the check means is a part of the guiding core that can be inserted into the guiding core tube and move forward within the guiding core tube; after moving out of the guiding core tube, check means of the guiding core can expand outward, and a span of the expanded check means is greater than an opening diameter, i.e. the moth span, of the cavity at the head end of the catheter.

Preferably, a liquid channel is arranged between the front end of the guiding core and the head end of the catheter.

Preferably, the cavity comprises one or more cavities which are provided in series.

According to the second aspect of the invention, there is provided a catheter, wherein a clamping means is provided at the front end of the guiding core to match with the head end of the catheter.

According to a third aspect of the invention, there is provided a catheter, wherein an internal cross-sectional area at a front end of the catheter for discharging urine can be fully used up to as large as an inner hole area of the catheter.

According to a fourth aspect of the invention, there is provided a catheter, wherein a head end of the catheter is directly connected with a guiding core.

Preferably, a front end of the guiding core is directly connected with the head end of the catheter.

Preferably, a front end of the guiding core is matched with the head end of the catheter.

Preferably, the check means is arranged directly within the head end of the catheter.

Preferably, a front end of the guiding core is matched with a protrusion of a head end of the catheter.

Preferably, the clamping means at the front end of the guiding core is matched with the head end of the catheter.

Preferably, a liquid channel is arranged between the front end of the guiding core and the head end of the catheter.

Preferably, the head end of the catheter is provided with a cavity, and an internal span of the cavity is greater than its mouth span.

Preferably, the cavity comprises one or more cavities which are arranged in series.

Preferably, the cavity accommodates a check means located at the front end of the guiding core.

Preferably, the check means is arranged at a tip of the guiding core that can retract into the guiding core tube and move along the guiding core tube.

Preferably, the guiding core can expand outward after moving out of the guiding core tube.

Preferably, the span of the expanded tip portion of the guiding core is greater than an opening diameter of the cavity arranged at the head end of the catheter.

Preferably, a limiting means is arranged at a tail end of the guiding core and/or the guiding core tube so as to regulate a position of the guiding core within the guiding core tube.

Preferably, a hardness of the head end is greater than that of the catheter body.

Preferably, the tail end of the catheter is provided with a connecting means, which is provided with a conical discharging hole and a lubricating fluid injecting hole.

Preferably, the connecting means is provided with an extra urine discharging hole, or the connecting means is provided with a channel leading to the urine discharging hole.

According to the invention, the head end of the catheter is directly connected with the guiding core, there is no longer any inner flange within the catheter at the guiding head thereof as provided in prior art, and there is no extra part arranged inside the front end of the catheter, that is, there is no need to add any accessory means within the catheter tube as does the traditional catheter. Therefore, it is no longer necessary to have to occupy some internal cross-sectional area of the catheter, so it is no longer to have to sacrifice some volume per minute for discharging urine by the catheter. Moreover, the resistance for injecting lubricant is much less than that of the prior art.

According to the invention, differing from the prior art, a cavity is independently arranged within the head end of the catheter or a cavity is formed by combining the upper and lower parts of the head end of the catheter; or a cavity is formed between a flange of the head end of the catheter and the inner wall of the catheter, the cavity can be composed of one or more cavities provided in series. The internal span of the cavity is greater than or equal to the span of the mouth (preferably greater than). The cavity can accommodate a check means or a clamping means. The check means is a head of the guiding core, the head can be expanded after entering the cavity. The clamping device is a portion of the guiding core, preventing the guiding core from withdrawing by clamping a hanging down column of the head end of the catheter after the head end of the guiding core enters the cavity.

According to the invention, there is provided the cavity which is not arranged in the head end of any traditional catheter, the cavity accommodates the check means or the clamping means, limiting any retraction of the guiding core within the guiding core tube in the catheter, so as to avoid any possibly damaging urethra due to such a risk that the guiding core would penetrate through the urine discharging hole of the catheter. Since the inventive cavity is provided to locate in front of any urine discharging hole in view of a longitudinal direction of the catheter from its tail end to its head end, the urine discharging efficiency of the catheter is not be sacrificed, and the cross-sectional area for injecting lubricant into the catheter is fully used to increase up to the whole inner cross section.

On the contrary, in prior art, an auxiliary structure is added within the catheter so as to prevent the guiding core from accidently passing through the urine discharging hole of catheter, so that the added auxiliary structure makes it needs a greater force to inject lubricant into the catheter, and lower the efficiency for discharging the urine. In particular, in prior art, in order to insert the catheter into the urethra of patients with urethral stricture or benign prostatic hyperplasia, a guiding wire is placed within the catheter to assist inserting the catheter into the urethra. However, clinically, the guiding wire needs to move forward or retract within the catheter. During movement within the catheter, the guiding wire might accidently pass through the urine discharging hole of the catheter, damaging or even piercing the patient's urethra.

According to the invention, any retracting displacement of the guiding core within the guiding core tube of the catheter is avoided, so that the guiding core are prevented from accidently penetrating through the urine discharge hole; at the same time, the lubricant injection can have a high efficiency, expanding the urethra, reducing a friction between the tube of the catheter and the urethra, thus reducing the pain of patients.

According to the invention, the front end of the guiding core is directly connected with the head end of the catheter, or the check means is directly installed on the tip of the catheter, so that the catheter is operated with an accurate guidance and an accurate lubrication during inserting into the urethra. As a result, the patients become comfortable when using the inventive catheter.

### BRIEF DESCRIPTION OF ACCOMPANYING DRAWINGS

FIG. 1 is a schematic view of a catheter that can smoothly pass through a urethral stricture section according to the present invention.

FIG. 2 is a schematic view of one embodiment of a head end of a guiding core according to the present invention.

FIG. 3 is a schematic view of another embodiment of the head end of the guiding core according to the present invention.

FIG. 4 is a schematic view of another embodiment of the head end of the guiding core according to the present invention when assembling is not completed.

FIG. 5 is a schematic view of another embodiment of the head end of the guiding core according to the present invention during being assembled.

### BEST MODES FOR CARRYING OUT THE INVENTION

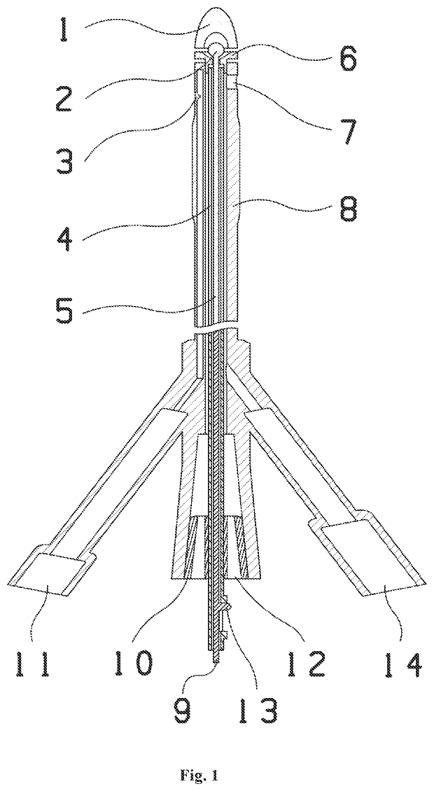

As shown in FIG. 1, according to an embodiment of the invention, an inventive catheter is a thin flexible tube which can be inserted into a human body, even smoothly passing through the narrowest section of urethra of the patient, so as to permit introduction of lubricant, or withdrawal of urine, or to keep a urine passageway open. The catheter includes: a head end upper part 1, a head end lower part 6, a catheter body 8, a guiding core tube head end 2, a guiding core tube 4, a guiding core 5, a liquid medicine inlet 11, a liquid medicine outlet 3, a conical urine draining hole 12 or a lubricant inlet 12, a flushing port 14 or a lubricant inlet 14, a urine draining hole or a lubricant outlet 7, a connecting means 10, a guiding core pushing end 9, and a guiding core stopper 13.

In one embodiment, the guiding core tube head end 2 is also called a check means, which is located at top of the guiding core 5, and can move back and forth in the longitudinal direction within the guiding core tube 4. When the guiding core 5 slides into a cavity arranged a larger inside but a smaller outside and located in the upper part 1 of the head end of the catheter, the guiding core tube head end 2 performs its check function to restrict any backward movement of the guiding core 5 within the guiding core tube 4, so that the guiding core 5 is prevented from moving backward within the guiding core tube 4, and then accidently passing through the urine discharging hole 7 or the liquid medicine outlet 3 on a side wall of the catheter body 8.

The guiding core stopper 13 is a limiting means of the guiding core 5, which limits any excessive propulsion of the guiding core pushing end 9 of the guiding core 5.

The connecting means 10 is arranged at the rear end of the catheter body 8, and fits between the inner wall of the catheter body 8 and the outer wall of the guiding core 5, so that the guiding core 5 can be centered inside the catheter body 8. At the same time, the connecting means 10 is also provided with an interface for connecting with the conical urine discharging port 12 at tail end of the catheter and an interface for connecting with a lubricant injecting device. The connecting means 10 is a pushing and locking means for the guiding core 5, and also a fixing means for the catheter tube 8. The connecting means 10 is also a means for guiding and controlling any forward and backward movement of the guiding core 5 within the guiding core tube 4. The connecting means 10 is provided with the conical urine discharging hole 12 and a urethra flushing hole 14. Alternatively, the conical urine draining hole 12 and the urethra flushing hole 14 can also be used as lubricant injecting holes. The connecting means 10 has a lubricant channel, which can be butted with any lubricant injecting device.

As shown in FIG. 2, in one embodiment, the check means, i.e., the guiding core head end 2, can be in a shape of a plurality of bones of an umbrella, can be folded or opened, can adopt multiple umbrella bones 21, or can be formed by bending an elastic metal wire 21 for many times. After the guiding core head end 2 of the guiding core 5 spreads out, the guiding core 5 remains within the guiding core tube 4. At this time, the guiding core head end 2 plays a check role, and the guiding core tube 4 plays a guiding role.

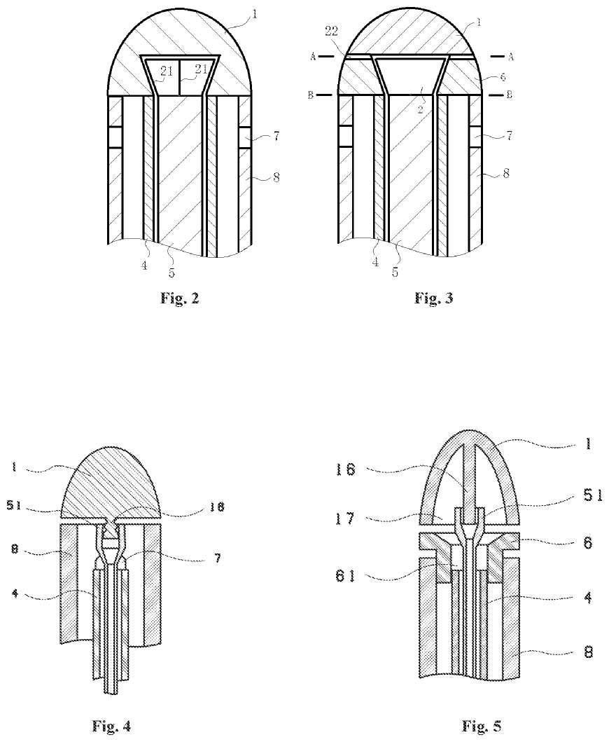

In the embodiment shown in FIG. 2, no small hole is provided in the head end of the catheter. In the embodiment shown in FIG. 3, one or more small holes 22 can also be arranged in the head end of the catheter, so as to increase an efficiency for discharging urine.

In the embodiment shown in FIG. 3, the head end of the catheter includes a head end upper part 1 and a head end lower part 6, and the head end upper part 1 and the head end lower part 6 can be bonded, inserted or clamped together. A small hole 22 is formed between the upper part 1 of the head end and the lower part 6 of the head end, for discharging urine from a urethra or injecting a lubricant into the urethra. The lower part 6 of the head end is provided with a funnel-shaped space, and the guiding core head end 2 is matched in the funnel-shaped space in order to form a check means.

The head end of the catheter can be bonded to the end of the catheter body 8, or bonded to the end of the guiding core tube 4 at the same time. For measuring a bonding strength, a separating force is tested at the end B of the catheter body 8 and at the position A where the small hole 22 is set at the head end of the catheter. According to the standard of the invention, when the outer diameter of the catheter is less than 3.3 mm, the separating force is more than 0.8 kg; while when the outer diameter of the catheter is ≥3.3 mm, the separating force is >1.1 kg.

According to the invention, any lubricant can be injected into a gap between the outer wall of the guiding core tube 4 and the guiding core 5, or another gap between the guiding core tube 4 and the inner wall of the catheter body 8, and can be forced to discharge from the urine discharging hole 7 and/or the small hole 22 at the guiding core head end, so as to be pushed to enter the urethra. The lubricant can also be injected from the inside of the guiding core tube 4, and the lubricant can be pushed to flow out from a side hole and/or top of the front end of the guiding core tube 4 into the urethra (FIG. 5). The lubricant can be injected through the liquid medicine injecting port 11, the urethra flushing port 14, or the conical urine discharging hole 12.

For the same type of catheter, the maximum diameter of the upper half 1 of the head end, the diameter of the lower half 6 of the head end, and the diameter of the catheter body 8 are the same as each other, preferably between 2-10 mm, and the thickness of the upper half 1 of the head end is not less than 0.1 mm. The bonding area between the lower half 6 of the head end and the end of the catheter body 8 is not less than the cross-sectional area of the tube wall of the catheter body 8.

According to the invention, the requirements for the minimum separating force (kg) between the head end upper half 1 and the lower half 6 and the minimum separating force (kg) between the head end lower half 6 and the catheter body 8 are as follows: when an outer diameter of the catheter body 8 is 2 mm or 2.7 mm, the minimum separating force (kg)>0.7, preferably >0.8; When the outer diameter of the catheter body 8 is 2.7 mm, 3.3 mm, 4 mm, 4.7 mm, 5.3 mm, 6 mm, 6.7 mm, 7.3 mm, 8 mm, 8.7 mm, 9.3 mm or 10 mm, the minimum separating force (kg) >1, preferably >1.1.

According to the invention, a catheter product can be used anytime within 60 months or more.

According to another embodiment of the invention, as shown in FIG. 4, the catheter head end 1 has a hanging down column 16 within the catheter head end, and the hanging down column 16 is integrated with the catheter head end 1, or installed on the catheter head end 1. There is a space between the hanging down column 16 of the catheter head end and the inner wall of the catheter body 8. A clamping means 51 arranged at the catheter head end is inserted into the space to clamp the hanging down column 16 of the catheter head end.

According to another embodiment of the invention, as shown in FIG. 5, the catheter head end is divided into an upper part 1 and a lower part 6. The upper part 1 has a hanging down column 16 of the catheter head end. The hanging down column 16 of the catheter head end is an integral structure with the upper part 1 of the catheter head end, or is installed on the upper part 1 of the catheter head end. A draining hole is arranged between the upper part 1 and the lower part 6. The lower part 6 is bonded to the end face of the catheter body 8. The lower part 6 has a central hole 61, and a clamping means 51 of the guiding core head end 2 of the guiding core 5 is inserted through the central hole 61 to clamp the hanging down column 16 of the catheter head end. There may be a gap between the lower part 6 and the guiding core 5 to allow the liquid to pass through. The lower part 6 and the guide core 5 can also be sealed. The lower part 6 includes a flange portion and a central tubular portion, the flange portion is bonded with the end face of the guiding core 5, while the central tubular portion is inserted into the guiding core tube 4 and bonded with the inner wall of the guiding core tube 4. In the upper part 1 of the catheter head end, a space 17 is arranged around the hanging down column 16 of the catheter head end so as to form a liquid turbulence, reduce the injected liquid impacting on the patient's urethra, and make the patient more comfortable.

A catheter according to the invention is characterized in that a head end of the catheter is provided with a cavity, an internal span of the cavity is greater than its mouth span, and the cavity accommodates a check means located at a front end of the guiding core.

The catheter according to the invention is characterized in that the head end of the catheter is directly connected with the guiding core.

The catheter according to the invention is characterized in that an internal cross-sectional area at the front end of the catheter for discharging urine is the same as the whole inner hole area of the catheter.

Preferably, the front end of the guiding core is directly connected with the head end of the catheter.

Preferably, the front end of the guiding core is matched with the head end of the catheter.

Preferably, the check means is arranged directly within the head end of the catheter.

Preferably, the front end of the guiding core is matched with a protrusion of the head end of the catheter.

Preferably, the clamping means at the front end of the guiding core is matched with the head end of the catheter.

Preferably, a liquid channel is arranged between the front end of the guiding core and the head end of the catheter.

Preferably, the head end of the catheter is provided with a cavity, and the internal span of the cavity is greater than the mouth span.

Preferably, the cavity comprises one or more cavities which are arranged in series.

Preferably, the cavity accommodates a check means located at the front end of the guiding core.

Preferably, the check means is a portion of the guiding core that can shrink into the guiding core tube and move within the guiding core tube.

Preferably, the guiding core can expand outward after moving out of the guiding core tube.

Preferably, a hardness of the head end is greater than that of the catheter body.

Preferably, the span of the expanded top guiding core is greater than an opening outer diameter of the cavity at the head end of the catheter.

Preferably, a limiting means is arranged at a tail end of the guiding core and/or the guiding core tube so as to regulate a position of the guiding core within the guiding core tube.

Preferably, the tail end of the catheter is provided with a connecting means, which is provided with a conical discharging hole and a lubricant fluid injecting hole.

Preferably, the connecting means is provided with a urine discharging hole, or the connecting means is provided with a channel leading to the urine discharging hole.

According to the invention, any extending portion of the guiding head, inserted into the catheter in the prior art, is eliminated, and any parts arranged in the front end of the catheter are avoided. There are no additional attachments provided on the inner surface of a tube wall of the catheter with respect to the prior art, so that the internal cross-sectional area of the catheter for discharging urine becomes the same as the inner hole area of the catheter, thereby greatly increasing the urine discharging volume of the catheter. Moreover, the injection resistance of lubricant is also greatly reduced.

Several embodiments of the present invention are disclosed above. According to the above disclosure, those skilled in the art can make various changes, modifications, improvements, or developments to the discussed embodiments. However, all the above activities will fall within the scope of protection as claimed in the appended claims.

1. A catheter, wherein within a head end of the catheter, there is provided with a cavity, a span of the cavity within its internal space is greater than that of a mouth of the cavity, and the cavity accommodates a check means located at a front end of a guiding core (preferably, the check means is a head portion of the guiding core, the head portion is operated to shrink into a guiding core tube and move forward within the guiding core tube; after moving out of the guiding core tube, the head portion of the guiding core expands outward, and a span of the expanded head portion of the guiding core is greater than a size of the mouth of the cavity at the head end of the catheter).

2. The catheter according to claim 1, wherein a liquid channel is arranged between a front end of the guiding core and the head end of the catheter (preferably, the liquid channel is arranged between a cylinder surface of the guiding core and an inner wall of the guiding core tube, and/or between an outer surface of the guiding core tube and an inner wall of the catheter).

3. The catheter according to claim 1, wherein the cavity comprises one or more cavities which are provided in series; or a hardness of the head end is greater than that of the catheter body.

4. A catheter, wherein a head end of the catheter is directly connected with a guiding core, and an internal cross-section area of a front end of the catheter for discharging urine is the same as the inner hole area of the catheter (preferably, the front end of the guiding core is integrated with the head end of the catheter; or the front end of the guiding core is matched with the head end of the catheter; or a check means is directly arranged within the head end of the catheter).

5. The catheter according to claim 4, wherein the front end of the guiding core is in engagement with a hanging down column of the head end of the catheter (preferably, a clamping means of the front end of the guiding core is matched with the head end of the catheter); and a hardness of the head end is greater than that of the catheter body.

6. The catheter according to claim 4, wherein a liquid channel is arranged between the front end of the guiding core and the head end of the catheter; or a hardness of the head end is greater than that of the catheter body.

7. The catheter according to claim 4, wherein the head end of the catheter is provided with a cavity, and a span of the cavity within its internal space is greater than that of a mouth of the cavity; preferably, the cavity comprises one or more cavities which are provided in series; alternatively, the cavity accommodates a check means located at the front end of the guiding core (preferably, the check means is a head portion of the guiding core, the check means is operated to shrink into a guiding core tube, move within the guiding core tube, and expand outward after moving out of the guiding core tube; a span of the expanded check means is greater than that of an opening of the cavity at the head end of the catheter).

8. The catheter according to claim 1, wherein at the end of the guiding core and/or the guiding core tube, there is provided with a limiting means, so as to regulate a position of the guiding core within the guiding core tube.

9. The catheter according to claim 1, wherein at the tail end of the catheter, there is provided with a connecting means, and the connecting means is provided with a conical hole for discharging urine and a hole for injecting lubrication.

10. The catheter according to claim 9, wherein the connecting means is further provided with a secondary hole for discharging urine, or the connecting means is provided with a channel communicating with the hole for discharging urine.
